(19) United States
(12) Patent Application Publication
(10) Pub. No.: US 2005/0112273 A1
(43) Pub. Date: May 26, 2005

(54) Method of improving the quality and performance of a coating on a coated medical device using a solvent to reflow the coating

(76) Inventor: Eric B. Stenzel, Galway (IE)

Correspondence Address: KENYON & KENYON, 1500 K STREET, N.W., SUITE 700, WASHINGTON, DC, 20005, US

(21) Appl. No.: 10/973,183

(22) Filed: Oct. 25, 2004

Related U.S. Application Data
(63) Continuation-in-part of application No. 10/440,141, filed on May 19, 2003.

(51) Int. Cl. A61F 2/02

(52) U.S. Cl. 427/2.1; 424/423

(57) ABSTRACT

A method is provided for coating at least a portion of at least one medical device. The method includes arranging a polymer on the portion of the medical device, arranging a bioactive agent on the portion of the medical device, and spraying, subsequent to the arranging of the polymer and the arranging of the bioactive agent, a solvent on the portion of the medical device. The method may further include selecting a composition of the solvent to achieve a desired agent release response profile for the medical device. A medical appliance is provided having a coating applied by a method that includes arranging a polymer on the portion of the medical device, arranging a bioactive agent on the portion of the medical device, and subsequently spraying a solvent on the portion of the medical device. A method is provided for achieving a desired agent release response profile for a medical device by reflowing the coating with a solvent.

### CROSS-REFERENCE TO RELATED APPLICATIONS

This application is a continuation-in-part of application Ser. No. 10/440,141 filed on May 19, 2003, which is incorporated by reference herein.

### FIELD OF THE INVENTION

The present invention relates to coating methods. More particularly, the present invention relates to a device and method for improving the coating quality and performance of a drug coated device such as a stent by respraying the coating with a solvent to reflow the coating.

### BACKGROUND INFORMATION

Medical devices may be coated so that the surfaces of such devices have desired properties or effects. For example, it may be useful to coat medical devices to provide for the localized delivery of therapeutic agents to target locations within the body, such as to treat localized disease (e.g., heart disease) or occluded body lumens. Localized drug delivery may avoid some of the problems of systemic drug administration, which may be accompanied by unwanted effects on parts of the body which are not to be treated. Additionally, treatment of the afflicted part of the body may require a high concentration of therapeutic agent that may not be achievable by systemic administration. Localized drug delivery may be achieved, for example, by coating balloon catheters, stents and the like with the therapeutic agent to be locally delivered. The coating on medical devices may provide for controlled release, which may include long-term or sustained release, of a bioactive material.

Aside from facilitating localized drug delivery, medical devices may be coated with materials to provide beneficial surface properties. For example, medical devices are often coated with radiopaque materials to allow for fluoroscopic visualization while placed in the body. It is also useful to coat certain devices to achieve enhanced biocompatibility and to improve surface properties such as lubriciousness.

Coatings have been applied to medical devices by processes such as dipping, spraying, vapor deposition, plasma polymerization, spin-coating and electrodeposition. Although these processes have been used to produce satisfactory coatings, they have numerous, associated potential drawbacks. For example, it may be difficult to achieve coatings of uniform thicknesses, both on individual parts and on batches of parts. Further, many conventional processes require multiple coating steps or stages for the application of a second coating material, or may require drying between coating steps or after the final coating step.

The spray-coating method has been used because of its excellent features, e.g., good efficiency and control over the amount or thickness of coating. However, conventional spray-coating methods, which may be implemented with a device such as an airbrush, have drawbacks. For example, when a medical device has a structure such that a portion of the device obstructs sprayed droplets from reaching another portion of the device, then the coating becomes uneven. Specifically, when a spray-coating is employed to coat a stent having a tube-like structure with openings, such as stents described in U.S. Pat. Nos. 4,655,771 and 4,954,126 to Wallsten, the coating on the inner wall of the tube-like structure may tend to be thinner than that applied to the outer wall of the tube-like structure. Hence, conventional spraying methods may tend to produce coated stents with coatings that are not uniform. Furthermore, conventional spraying methods are inefficient. In particular, generally only 5% of the coating solution that is sprayed to coat the medical device is actually deposited on the surface of the medical device. The majority of the sprayed coating solution may therefore be wasted.

In the spin-dipping process, a medical device is coupled to a spinning device, and then, while rotating about a central axis, the medical device is dipped into a coating solution to achieve the desired coating. This process also suffers from many inefficiencies including the unevenness of the coated layer and a lack of control over the coated layer's thickness.

In addition to the spray coating and spin-dipping methods, the electrostatic deposition method has been suggested for coating medical devices. For example, U.S. Pat. Nos. 5,824,049 and 6,096,070 to Ragheb et al. mention the use of electrostatic deposition to coat a medical device with a bioactive material. In the conventional electrodeposition or electrostatic spraying method, a surface of the medical device is electrically grounded and a gas may be used to atomize the coating solution into droplets. The droplets are then electrically charged using, for example, corona discharge, i.e., the atomized droplets are electrically charged by passing through a corona field. Since the droplets are charged, when they are applied to the surface of the medical device, they will be attracted to the surface since it is grounded.

One disadvantage of conventional electrostatic spraying is that it requires a complicated spraying apparatus. In addition, because conventional electrostatic systems use a gas to move the droplets from a source to a target, controlling the gas pressure is crucial for accurate coating. However, it is not easy to control the gas pressure so that the target surface is evenly and sufficiently coated without losing much of the coating solution.

Another method of coating a device can be achieved with electrohydrodynamic spraying. Using this method, a gas is not needed to disperse the coating. Electrohydrodynamic coating is accomplished by forcing a compatible solution through a nozzle assembly that has been electrically charged. The coating solution passes through the charged nozzle where it is electrically charged. As the solution exits the nozzle, the solution atomizes as the charged particles repel each other. This action forms the spray mist. The charged particles are attracted to the device to be coated since the device is connected to an electrical ground.

Devices may be coated by a gas assisted spraying process. A polymer/drug combination may be dissolved in a solvent mixture. The solution may be sprayed onto the devices and a polymer/drug film may be formed when the solvents evaporate. The ability to apply thin coatings on products may be limited by the capabilities of a gas assisted spraying process. The coating may flow on the medical device prior to drying, thereby creating an uneven concentration of bioactive agent on the surface of the device. A gas assisted spraying process may have a high variability for thin coatings.

Conventional methods of coating stents or devices with a drug-polymer layer, such as spraying or dipping, may require a solution of the drug-polymer to physically wet the surface of the stent. Spraying or dipping may cause uneven and unpredictable wetting, and distribution and evaporation of the solvent molecules may result in a non-uniform coating. The drying of the coating may lead to cracking and/or points of stress in the coating. A non-uniform coating may lead to the unit failing agent release requirements, drug uniformity and coating thickness specifications.

There is, therefore, a need for a cost-effective method of coating devices that results in uniform, defect-free coatings and uniform drug doses per unit device. The method would provide better control of the agent release profile of the device, including increasing or decreasing the release of the bioactive agent. The method would also improve the quality of the coating of the device by removing defects, cracks and stress points in the coating. The method would thus allow for better control of the sensitivity of the bioactive material and would reduce variations in the coating properties. Each of the references cited herein is incorporated by reference herein for background information.

### SUMMARY

A method is provided for coating at least a portion of at least one medical device. The method includes arranging a polymer on the portion of the medical device, arranging a bioactive agent on the portion of the medical device, and spraying, subsequent to the arranging of the polymer and the arranging of the bioactive agent, a solvent on the portion of the medical device. The polymer may include a polymer compound such as SIB. The bioactive agent may include Ptx. The solvent may include at least one member chosen from a group consisting of: tetrahydrofurane (THF), toluene, dimethylacetamide (DMACE), acetone, chloroform, and alcohol.

The arranging of the polymer may include spraying the polymer in a liquid form on the medical device. The arranging of the bioactive agent may include dusting the medical device with a dry powder that may include some or all of the bioactive agent. The arranging of the bioactive agent may be performed subsequent to the arranging of the polymer. The method may further include drying the polymer on the medical device prior to the arranging of the bioactive agent by dusting. The drying may be achieved by at least one of waiting a predetermined time period, heating the medical device, and blowing a gas on the medical device. The arranging of the bioactive agent by dusting may be performed while the polymer is wet. The spraying of the solvent on the portion of the medical device may dissolve the dry powder including the bioactive agent. The spraying of the solvent on the portion of the medical device may dissolve the polymer. The arranging of the polymer and the arranging of the bioactive agent may be performed simultaneously by spraying the medical device with a solution including the bioactive agent and the polymer. The solution including the bioactive agent and the polymer may be allowed to dry before the subsequent spraying of the solvent on the portion of the medical device. The subsequent spraying of the solvent on the portion of the medical device may cause the dried solution to reflow. The subsequent spraying of the solvent causing the dried solution to reflow may reduce stress in the dried solution and/or smooth a surface of the dried solution. The subsequent spraying of the solvent may re-position the polymer or bioactive compounds in the coating layer from where they were originally deposited by moving one compound closer to or further from the surface.

The method may further include selecting a composition of the solvent to achieve a desired agent release profile for the medical device. The composition of the solvent may be selected to achieve an increased or a decreased agent release profile for the medical device. A concentration of THF may be increased, thereby causing the bioactive agent to migrate to a surface of the coating to increase the agent release profile of the device. A concentration of toluene may be increased, thereby causing the polymer to migrate to a surface of the coating to decrease the agent release profile of the device.

A medical appliance is provided having a coating applied by a method that includes arranging a polymer on the portion of the medical device, arranging a bioactive agent on the portion of the medical device, and subsequently spraying a solvent on the portion of the medical device. The method may further include selecting a composition of the solvent to achieve a desired agent release profile for the medical appliance. The composition of the solvent may be selected to achieve an increased agent release profile for the medical device. A concentration of THF may be increased, thereby causing the bioactive agent to migrate to a surface of the coating. The composition of the solvent may be selected to achieve a decreased agent release profile for the medical device. A concentration of toluene may be increased, thereby causing the polymer to migrate to a surface of the coating.

### BRIEF DESCRIPTION OF THE DRAWINGS

FIG. 1 is a schematic representation of an exemplary embodiment of the invention.

FIG. 2 is a flowchart for performing an exemplary method of the invention.

FIG. 3 is a graphical illustration comparing an agent release profile for a stent that has been subjected to an exemplary method according to the present invention with a conventionally coated stent.

FIG. 4 is a graphical illustration comparing an agent release profile for a stent that has been subjected to an exemplary method according to the present invention with a conventionally coated stent.

FIG. 5 is a graphical illustration comparing an agent release profile for a stent that has been subjected to an exemplary method according to the present invention with a conventionally coated stent.

FIG. 6 is an enlarged view of a coated stent showing struts and a junction before using an exemplary method of the present invention.

FIG. 7 is an enlarged view of the coated stent of FIG. 6 after using an exemplary method of the present invention.

FIG. 8A shows atomic force microscopy imaging of a conventionally coated device.

FIG. 8B shows atomic force microscopy imaging of a tetrahydrofurane reflowed device.

FIG. 9 is a chart illustrating that a respray with a solvent increased the therapeutic concentration at and/or near the surface of a device.

### DETAILED DESCRIPTION

In an exemplary method according to the present invention, the device that has been coated is sprayed with a solvent and/or solvent mixture in order to reflow the coated layer and create a final surface finish. The reflow following the spraying with the solvent-only mixture may yield a coating that is uniform and/or consistent, which may be independent of the spray parameters used to coat the device originally with the drug-loaded polymer compound.

This process may be added as an additional step after the device is coated and dried, or may be completed immediately after the polymer/drug compound is applied (for example, while the polymer/drug compound is still wet).

After a device has been sprayed with a solvent-only solution, the polymer/drug layer surface finish may be more consistent from batch to batch irrespective of the coating parameters used to apply the coating.

Additionally, there may be added benefits where the selection of solvents used have the effect of controlling the amount of Ptx that may ultimately reside at or near the surface of the coating. For example, the selected solvent(s) may either draw the Ptx towards the surface of the coated layer and/or drive the Ptx down below the surface of the coated layer.

In an alternative exemplary embodiment of the present invention, a polymer-only coated device may be sprayed, dusted, coated and/or otherwise covered with a dry drug compound powder, and a solvent re-spray process may then be used to integrate the drug into the polymer layer. In this exemplary embodiment, the re-spray process may not be a method or process to recondition a coating but may instead be a method of combining the drug to the polymer. In this exemplary method, the drug may be positioned near the outer surface of the polymer, where it may be most useful. In this alternative exemplary embodiment, the process would be to first coat with a polymer only, then to infuse a drug into the coating by dusting with a drug, and then to spray with a solvent only.

As used herein, the term “therapeutic agent” includes one or more “therapeutic agents” or “drugs”. The terms “therapeutic agents”, “active substance” and “drugs” are used interchangeably herein and include pharmaceutically active compounds, nucleic acids with and without carrier vectors such as lipids, compacting agents (such as histones), virus (such as adenovirus, andenoassociated virus, retrovirus, lentivirus and α-virus), polymers, hyaluronic acid, proteins, cells and the like, with or without targeting sequences.

In one exemplary embodiment of the invention, a process for coating a structure such as a stent may include first preparing a mixture of a polymer and an active substance or therapeutic. The mixture may be in the form of a powder. Then, the exemplary process includes forming a first coating on the structure by electrostatically depositing the mixture on the structure. This step can be followed by a step to cure the mixture.

One method of curing the mixture includes forming a second coating to at least partially cover the first layer. The second coating can include a solvent. If a second coating including a solvent is deposited, the coating can be applied such that at least portions of the first coating are covered by the solvent. The solvent can be evaporated to enable the reflow of the first coating. Alternatively, the second coating of one or more solvents is formed on, and is absorbed by, the first coating to cause coating reflow. The solvents can be evaporated from the device by using infrared drying, laser drying, convection of hot air, vacuum ovens, or a combination of temperature, pressure, and solvent vapor pressure.

In an alternative embodiment, the mixture can be cured by heating the powder covered stent to melt the first coating, causing it to flow. This can be used with or without a second coating.

The step of forming a first coating or the first layer on the structure can be implemented in accordance with one of several methods. In one embodiment of the invention, the first coating is deposited and the first layer is formed on the structure through electrostatic deposition. According to this embodiment, a mixture containing the desired ratio of an active substance and one or more polymers is prepared. The active substance(s) and the polymer can be ground and/or milled to form a fine powder. The active substance(s) and/or the polymer can be chilled as desired to freeze the material into a solid to enhance the process of forming a powder through grinding and/or milling. While powder size of any suitable range can be used, in one embodiment, the polymer and the active substance are milled to form a fine powder in the range of 0.0001-0.025 mm.

If the polymer or the active substance is a liquid at room temperature, it can be frozen with liquid nitrogen or other suitable freezing method to assume a solid phase. Once the mixture or one of the ingredients has been prepared in the frozen state, it can be ground and deposited in the frozen state. The frozen material may also be a combination of bioactive materials and polymers. The electrostatic coating system can comprise any conventional coating system that uses electrostatic deposition principles.

In another embodiment, the invention defines a process for coating a medical device, the process including forming a first layer on a structure where the first layer includes a bio-compatible polymer; forming a second layer on the structure where the second layer includes an active substance; exposing at least one of the first or the second layers to a solvent to reflow the active substance; and drying the structure. The step of forming a first layer on the structure can further include grinding the active substance into a fine powder, electrostatically charging the fine powder, discharging the structure to substantially free the structure from electrostatic charge and depositing the fine powder on the structure. A medical device coated in this manner can be inserted into a body lumen.

In one embodiment of the invention, the powder is passed through an electromagnetic field (e.g., corona discharge) having a flux density for charging the powder particles. Once the particles are charged, they can be directed toward the structure. The structure can be grounded so as to have no charge. Alternatively, the structure can be charged to have an opposite charge to that of the powder particles. In either case, the attraction between the charged particles and the grounded structure will draw the particles to the structure. Once the powder particles are placed near the structure, the electromagnetic field will transport the powder particles onto the structure. As charged powder particles are deposited on the surface of the structure, a coating is formed. The areas not covered by the coating will retain their greater attraction for the charged particles. During the coating process, the charged particles will seek out the greatest attractive force. At the start of the powder coating process, the greatest force is associated with the bare metal frame; hence this area will coat first. As a result, the newly introduced particles will be drawn to areas that have not been coated. As the bare metal areas become covered in the powder mixture, the next most attractive surface would be the thinnest coated area on the device. This provides for an even coating of the device. Thus, a coating layer of substantially uniform thickness will form throughout the surface of the structure.

Additional steps can be taken to ensure that the desired amounts of the active substance and/or the polymer have been deposited. For example, the structure can be coupled to a fine scale that monitors the weight of any additional coating deposited thereon. In this embodiment, the electrostatic deposition process continues until the stent and the coating reach the desired weight.

In another embodiment the invention the powder is passed through a conductive nozzle that has been charged to a high voltage. As the powder passes through the nozzle, an electric charge is transferred to the powder. As the powder leaves the nozzle, the powder particles will repel from adjacent charged particles while being drawn through the electric and magnetic field created between the charged nozzle and the grounded device to be coated.

In one embodiment of the invention, the first layer comprises either a binder or an active substance. According to this embodiment, once the first layer has been deposited, a second layer can be deposited to include the missing ingredient(s). The second layer can include one or more solvents.

Once the combination of an active substance and one or more polymers have been deposited to form a coating, a reflow of the coating can be initiated. Reflow can be initiated by heating the structure with the coating(s) thereon to cause melting of the coating(s). For example, the structure can be placed in an oven or a heated chamber to melt the polymer coating and cause reflow thereof. Once the polymer coating is melted it reflows to cover the surface of the structure. In an embodiment where the structure is a stent, the reflow will enable the coating to substantially cover the struts.

Reflow can also be initiated by adding a coating of one or more solvents. The solvent may be sprayed on the coated stent to cause the polymer to reflow. In another embodiment, solvent can be electrostatically charged and then deposited on the structure to at least partially cover any existing coating(s). Suitable solvents that can be sprayed or electrostatically deposited on the structure include tetrahydrofurane (THF), chloroform, toluene, methyl ethyl keton (MEK), DMACE, acetone, alcohol and any other solvent that may be sprayed or electrostatically deposited.

In another exemplary embodiment, solvent is added by dipping the structure into the solvent.

In still another exemplary embodiment, the pre-coated structure is placed in a chamber having a high vapor pressure of a solvent at a first temperature. A pre-coated device at a second temperature that is lower than the first temperature is lowered into solvent vapor. The solvent vapor will condense on the cooler pre-coated device thus transfer solvent to the pre-coated device causing the polymer to absorb the solvent and reflow to form the coated layer.

The coating material can comprise an active substance in a polymer matrix. According to one embodiment, an active substance is dissolved in a polymer solution to form a liquid mixture. The liquid mixture can be crystalized by any of the conventional methods to form a powder. A preferred method of crystallizing the mixture may be to freeze it. Next, the powder is ground to a size suitable for electrostatic deposition and deposited on the structure.

Curing the mixture can occur in-situ. To facilitate curing, a cross-linking or curing agent may be added to the mixture prior to application thereof. Addition of the cross-linking or curing agent to the polymer/drug agent liquid mixture must not occur too far in advance of the application of the mixture in order to avoid over-curing of the mixture prior to application thereof. Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

In an exemplary embodiment, the polymer used to coat the medical device is provided in the form of a coating on an expandable portion of a medical device. After applying the drug solution to the polymer and evaporating the volatile solvent from the polymer, the medical device is inserted into a body lumen where it is positioned in a target location. In the case of a balloon catheter, the expandable portion of the catheter can be subsequently expanded to bring the drug-impregnated polymer coating into contact with the lumen wall. The drug is released from the polymer as it slowly dissolves into the aqueous bodily fluids and diffuses out of the polymer. This enables administration of the drug to be site-specific, limiting the exposure of the rest of the body to the drug.

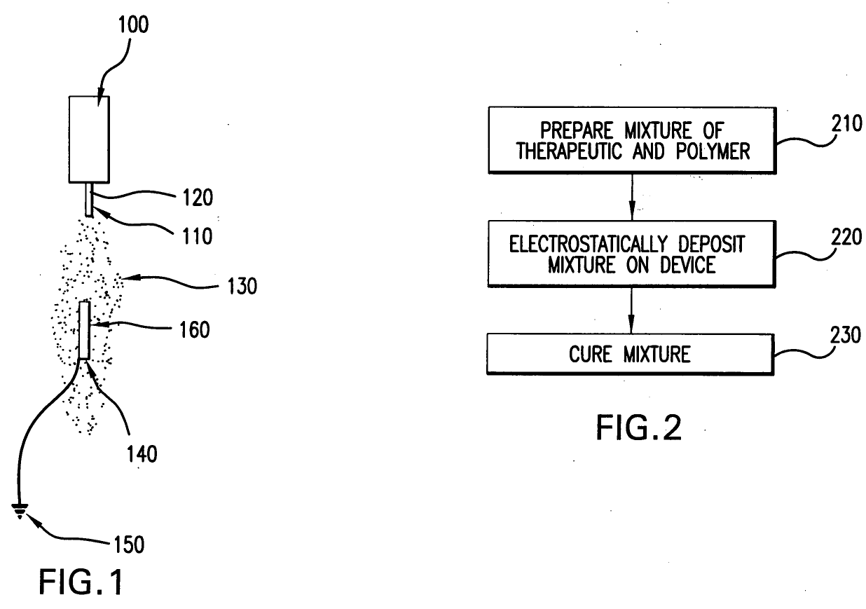

FIG. 1 is a schematic representation of one embodiment of the invention. FIG. 1 shows powder application unit 100 that pumps the powder through conductive charging nozzle 110. Depending on the application, the powder application unit may need to be cooled to retain the powder in a solid state. In the embodiment of FIG. 1, high voltage power source 120 is connected to conductive charging nozzle 110 to provide electrostatic charge thereto. Charged powder stream 130 exits the nozzle and is immediately repelled from similarly-charged particles. This causes the charged particle stream to disperse as a cloud. The device to be coated, in this case stent 160, can be placed in the proximity of charging nozzle 110. As shown in FIG. 1, stent 160 is grounded through wire 140 to ground point 150. Ground point 150 need not be an absolute grounding point. Rather, it may be sufficient that ground point 150 have zero potential with respect to high voltage source 120, thus causing electrostatic attraction of charged powder stream 130 (now dispersed into a charged powder cloud) to stent 160.

FIG. 2 illustrates general categories of steps that may be undertaken to carry out the invention. Referring to FIG. 2, a mixture of therapeutic and polymer material is prepared at step 210. At step 220, the mixture is then electrostatically deposited on the device that is to be coated. Finally, the coated mixture is cured at step 230.

A study of re-spraying coated stents with pure THF provides data concerning the impact on agent release requirements due to re-spraying a PTx coated stent with pure THF. The experiment tested whether re-wetting a coated stent with pure THF would draw the PTx towards the surface as the solvent exited, and showed that the agent release response may increase with the PTx closer to the surface of the stent.

A batch of 24 stents was processed as normal to deposit a coating containing a combination of solvents, polymer and therapeutic. These stents were then dried in an oven. 12 stents were then reprocessed using the same coating conditions but applying a coating of pure THF instead of the previous combination.

After THF respray, these 12 stents were again dried in an oven and then processed to completion with the normally processed stents. This provided 12 control stents that were coated as per normal coating process and 12 stents that were processed with the THF re-spray steps added.

These stents were then tested to determine the agent release response for both the normal coating process conditions and the new process that included the THF re-spray.

The results of the experiment show that the initial burst release of the kinetic drug release test may be substantially increased. With this increase, a component stent that may have previously failed a batch release specification may now pass.

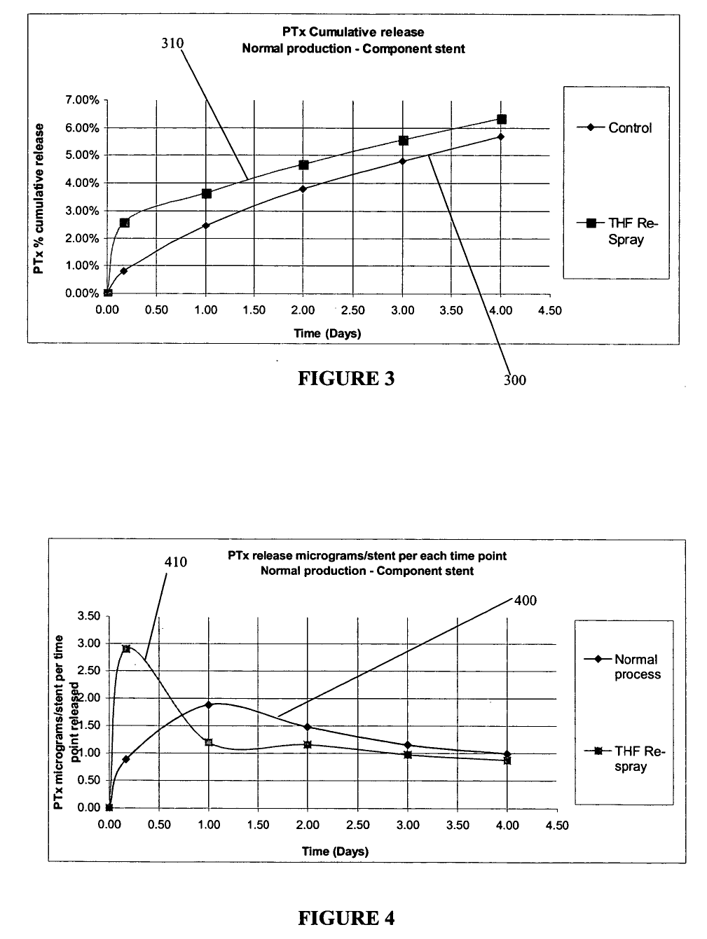

FIG. 3 shows a graph comparing cumulative agent release response. Curve 300 represents a best-fit line for the control group of stents. Curve 310 represents a best-fit line for the THF respray group of stents. As can be seen in FIG. 3, there is a substantial increase in the initial burst release of PTx from the re-sprayed stent. The data used to complete FIG. 3 is shown below as table 1 along with a percentage change.

Table 1 compares % PTx release:

TABLE 1

| | Normal process | THF re-sprayed | |
|---|---|---|---|
| Time point | % cumulative release | % cumulative release | % Change |
| 4 hour | 0.76% | 2.54% | +234% |
| 1 Day | 2.36% | 3.63% | +54% |
| 2 Day | 3.61% | 4.69% | +30% |
| 3 Day | 4.82% | 5.57% | +15% |
| 4 Day | 5.70% | 6.35% | +11% |

Table 1 highlights the substantial initial burst release that the THF re-spray has created on the coated stent. A comparison of the drug that is released at each time point shows the effect that the THF re-spray has on the drug release profile.

FIG. 4 shows a graph comparing PTx release (micrograms/stent) at each time point. Curve 400 represents a best-fit line for the stents prepared using a conventional process. Curve 410 represents a best-fit line for the THF re-spray group of stents. FIG. 4 highlights the extent of the PTx burst at the four-hour point and shows that the release points later in time begin to converge with each other.

In addition to the described agent release response improvements, the relative standard deviation of the re-sprayed stents may be approximately one-half for the standard coated batch. Thus, this initial experiment also shows that a THF re-spray may improve agent release response capability. This is shown in table 2 below.

Table 2 illustrates the impact of THF re-spray to relative standard deviation of agent release response results (“relative standard deviation” being written as RSD):

TABLE 2

| | Normal process | THF Re-spray | |
|---|---|---|---|
| Time (days) | RSD | RSD | % Change |
| 0.17 | 3.5% | 2.3% | −35% |
| 1 | 3.3% | 1.4% | −56% |
| 2 | 3.0% | 1.4% | −53% |
| 3 | 2.8% | 1.6% | −43% |
| 4 | 2.8% | 1.7% | −40% |

The re-spray with pure THF may draw the PTx towards the surface as the solvent dries from the stent. THF is the component that dissolves the PTx in the solution while the toluene dissolves the SIBs. When a stent is re-sprayed with pure THF, the PTx may be drawn towards the solvent. As the solvent dries and exits the surface of the coating, the PTx continues to be drawn towards the THF and may therefore be transported towards the surface of the coating.

FIG. 4 apparently shows that the PTx release of a normal batch and a THF re-sprayed batch seem to converge.

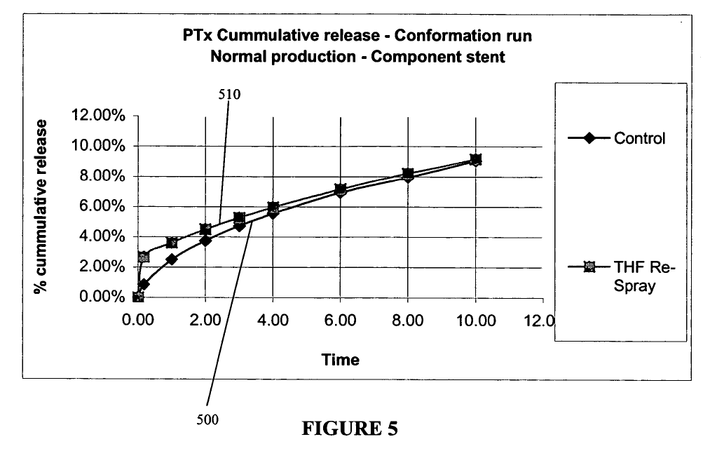

A conformation run was completed that confirmed these results. FIG. 5 illustrates a agent release response from a conformation run. Curve 500 represents a best-fit line for the control group of stents. Curve 510 represents a best-fit line for the THF re-spray group of stents. The two agent release response curves appear to converge over the extended test period of this agent release response test for the conformation run.

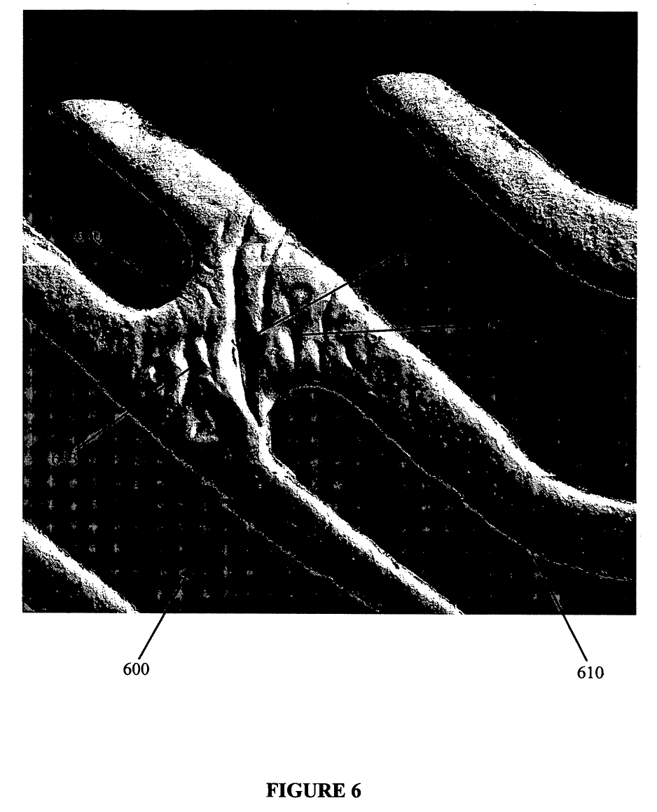

FIG. 6 is an enlarged view of coated stent 600 showing struts 610 and junction 620 a junction before using an exemplary method of the present invention using a confocal microscope. Stent 600 includes struts 610 connected by junctions 620. The coating on stent 600 has been applied in a conventional manner and, during the drying process, depressions 631, 632, 633 have developed. Additionally, cracks and ridges may develop in a coating during the coating and/or drying process. Depressions, 631, 632, 633, as well as cracks and ridges, may represent points of stress in the coating, and therefore points of weakness in the coating. Depressions 631, 632, 633 may lead to cracking, which may lead to flaking of the coating, an unwanted result. Additionally, depressions 631, 632, 633 may represent areas of the coating with less coating and therefore possibly less bioactive ingredient.

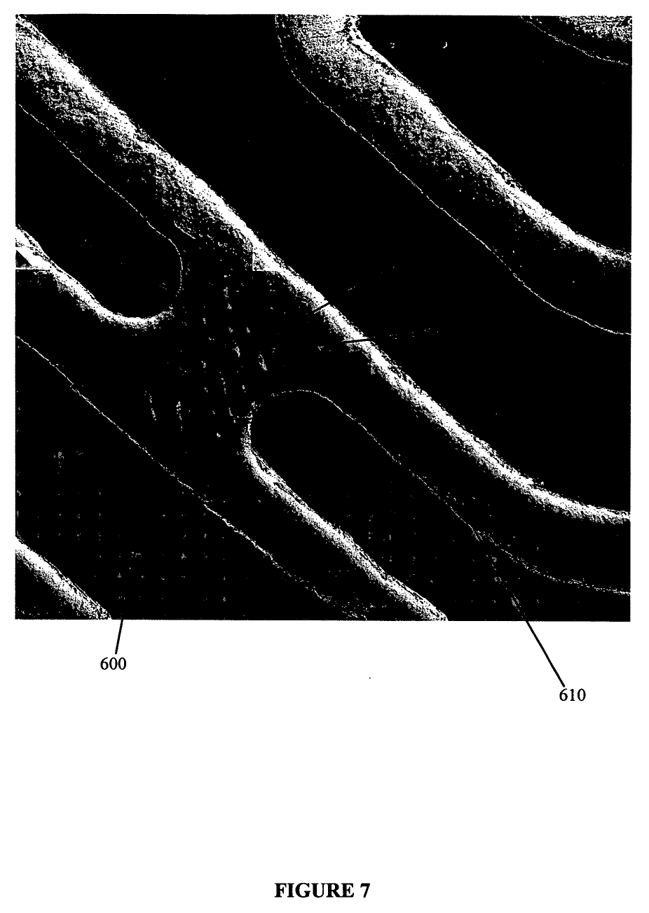

FIG. 7 is an enlarged view using a confocal microscope of coated stent 600 of FIG. 6 after using an exemplary method of the present invention. FIG. 7 shows that the THF re-spray may have smoothed the surface of the coating, as is apparent from a comparison of reflowed depressions 701, 702, 703, with depressions 631, 632, 633 of FIG. 6. This may also be construed as a form of stress relief of the coating layer where the coated layer is allowed to reflow using the THF re-spray process. FIG. 7 shows that the coating over junction 620 connecting struts 610 on stent 600 after the THF re-spray is smoother than the coating on stent 600 in FIG. 6 before the THF re-spray. Alternatively, the change in surface roughness may be attributed to a coating reflow that may not result in stress relief. A coating reflow that does not relieve internal stress in the coating may improve the strength and integrity of the coating and may improve other surface properties of the coating (for instance, lubriciousness).

Therefore, the THF reflow process may provide a method of increasing the agent release response of coated stents and may provide a method of reworking existing coated stents that have failed agent release response testing.

The solvent reflow process may also provide a method for repositioning the therapeutic agent within the polymer carrier. For example, a conventional coating process may apply a homogeneous coating where the therapeutic agent is evenly dispersed within the polymer carrier. The solvent reflow process provides a means for redistributing the therapeutic agent within the polymer carrier.

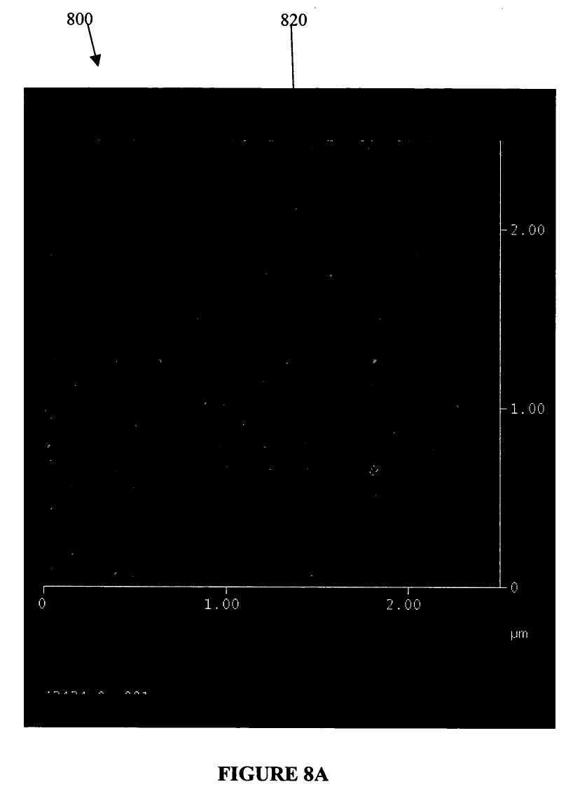

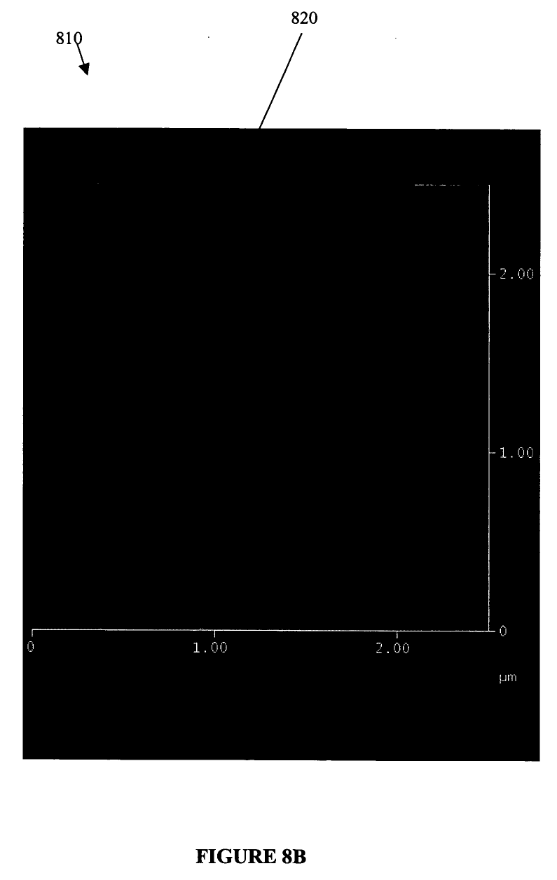

FIG. 8A below shows atomic force microscopy (AFM) imaging of surface 800 of a normally coated device while FIG. 8B shows AFM imaging of surface 810 a THF reflowed device. In FIGS. 8A and 8B, lighter colored elements 820 of the images shows the therapeutic agent at surface 800, 810 of the coating layer. As can be seen in FIG. 8B, the amount of lighter colored elements 820 appears denser than FIG. 8A.

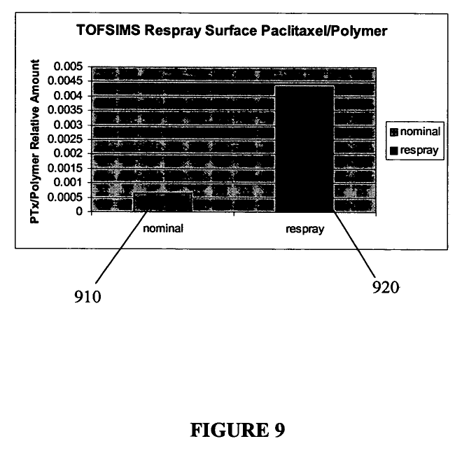

A TOFSIMS (Time of Flight Secondary Ion Mass Spectrometry) analysis was conducted to determine the ratios of paclitaxel (the therapeutic) to an element in the polymer carrier to ensure that the images in FIGS. 8A and 8B properly reflect that the images show a greater concentration of therapeutic at and near the surface after respraying. FIG. 9 shows that respray with a solvent increases the therapeutic concentration at and near the surface by a factor of approximately eight over conventionally coated surface. Conventional coating ratio 910 is approximately eight times respray ratio 920.

As can be seen visually from the AFM images in FIGS. 8A and 8B, the concentration at or near surface 810 of the coating has increased over surface 800 following the solvent respray. Furthermore, as can be seen from the TOFSIMS analysis summary in FIG. 9, the ratio of paclitaxel to a component of the polymer carrier has increased after respraying.

In an alternative exemplary embodiment, the agent release response may be decreased if the coated stent is sprayed with pure toluene and/or the distribution of the therapeutic within the polymer carrier can be altered to decrease the concentration of the therapeutic at or near the surface of the coating.

The therapeutic agent may be any pharmaceutically acceptable agent such as a non-genetic therapeutic agent, a biomolecule, a small molecule, or cells.

Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, epothilone, cladribine, 5-fluorouracil, methotrexate, doxorubicin, daunorubicin, cyclosporine, cisplatin, vinblastine, vincristine, epothilones, endostatin, trapidil, halofuginone, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis (2-aminoethyl)ethyleneglycol-N,N,N′,N′-tetraacetic acid and mixtures thereof; antibiotics such as gentamycin, rifampin, minocyclin, and ciprofolxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, warfarin sodium, Dicumarol, aspirin, prostaglandin inhibitors, platelet aggregation inhibitors such as cilostazol and tick antiplatelet factors; vascular cell growth promotors such as growth factors, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; inhibitors of heat shock proteins such as geldanamycin; and any combinations and prodrugs of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include monocyte chemoattractant proteins (“MCP-1) and bone morphogenic proteins (“BMP's”), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homdimers, heterodimers, or combinations thereof, alone or together with other molecules. Alternatively, or in addition, molecules capable of inducing an upstream or downstream effect of a BMP can be provided. Such molecules include any of the “hedghog” proteins, or the DNA's encoding them. Non-limiting examples of genes include survival genes that protect against cell death, such as anti-apoptotic Bcl-2 family factors and Akt kinase and combinations thereof. Non-limiting examples of angiogenic factors include acidic and basic fibroblast growth factors, vascular endothelial growth factor, epidermal growth factor, transforming growth factor α and β platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (“TK”) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered.

Any of the therapeutic agents may be combined to the extent such combination is biologically compatible.

Any of the above mentioned therapeutic agents may be incorporated into a polymeric coating on the medical device or applied onto a polymeric coating on a medical device. The polymers of the polymeric coatings may be biodegradable or non-biodegradable. Non-limiting examples of suitable non-biodegradable polymers include polystrene; polyisobutylene copolymers and styrene-isobutylene-styrene block copolymers such as styrene-isobutylene-styrene tert-block copolymers (SIBS); polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L,-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodable polymer such as polyhydroxybutyrate and its copolymers, polycaprolactone, polyanhydrides (both crystalline and amorphous), maleic anhydride copolymers, and zinc-calcium phosphate.

In a preferred embodiment, the polymer is polyacrylic acid available as HYDROPLUS® (Boston Scientific Corporation, Natick, Mass.), and described in U.S. Pat. No. 5,091,205, the disclosure of which is incorporated by reference herein. In a more preferred embodiment, the polymer is a co-polymer of polylactic acid and polycaprolactone.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

The coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers. Methods of choosing the type, thickness and other properties of the polymer and/or therapeutic agent to create different release kinetics are well known to one in the art.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, lung, liver, heart, skeletal muscle, kidney, bladder, intestines, stomach, pancreas, ovary, cartilage, eye, bone, and the like.

A dry powder containing polymers therapeutics may be electrostatically deposited on a device. The device may then be placed in an atmosphere with a high content solvent vapor pressure in a process similar to vapor phase cleaning. The solvent vapor may wet the coated device, dissolve, and reflow the deposited powder to form the coating.

In an exemplary embodiment of the invention, a first coating can be prepared by preparing a fine powder mixture of paclitexal and one or more polymer; alternatively, the mixture can include a mixture of a polymer, paclitaxel and a solvent. The mixture can be grinded and charged to have an electrostatic charge. The charged mixture can be deposited on a structure using the appropriate electrostatic deposition equipment. To enhance deposition, the structure can be grounded. In addition, the structure can be connected to a micro-scale that monitors the weight of the deposited coating. Thereafter, the structure having one layer of coating can be placed in a chamber that has a high vapor pressure of the solvent present. The presence of solvent and its deposition on the coating can initiate the reflow process. Optionally, after the electrostatic process is completed, air brush or other means can be used to remove any loose particles or coating.

It is envisioned that the above method may be utilized wherein the first coating does not include a solvent. Alternatively a solver/polymer/therapeutic mixture can be prepared, then frozen solid, then ground as a powder, and then electrostatically deposited. When the coating melts, the coating will reflow.

The present invention concerns bio-compatible medical devices and process for preparation thereof. The process includes electrostatically forming a first layer on a structure. The first layer can include a combination of at least one active substance and at least one polymer and/or binder. A second layer is formed on the structure to cover the first layer. The second layer can be a solvent or a combination of solvents. The evaporation of the second layer causes the first layer to reflow and bind to the structure.

The present invention concerns methods and apparatus for providing a substantially uniform coating on a structure. In one embodiment, the present invention is directed to a medical device adapted for insertion into a body lumen wherein the medical device is coated with an active substance and a bio-compatible polymer for binding the active substance to the structure.

In a process according to one embodiment of the invention, a method for coating a medical device includes forming a first coating on a bio-compatible structure by electrostatically depositing an active substance and a polymer on the structure, forming a second coating on the structure by depositing a solvent to at least partially cover the first coating, and causing a reflow of the first coating by evaporating the solvent from the structure. The polymer can be a binder or a resin or any material that can bind the active substance on the structure.

In a process according to another embodiment of the invention, a medical device is coated by forming a first layer of an active substance on a structure, forming a second coating to at least partially cover the first coating, the second coating having a bio-compatible polymer, and exposing at least one of the first or the second coating to a solvent to cause a re-flow the polymer. The step of forming a first coating on the structure can include grinding and/or milling the active substance into a fine powder and electrostatically charging and depositing the powder on the structure using the electrostatic charge difference between the active substance and the structure.

A process for coating a medical device includes forming a first coating on a bio-compatible structure by electrostatically depositing a mixture of a biologically active substance and a polymer on the structure; forming a second coating on the structure by depositing a solvent to at least partially cover the first coating; and evaporating the second coating to cause a reflow of the first coating over the structure.

The polymer may be a binder. The process may further include freezing the active substance to form a solid phase, grinding and/or milling the solid phase to form a frozen solid powder and mixing the frozen solid powder with the polymer to form the mixture of the active substance and the polymer.

The polymer may be frozen to a solid phase, ground to a powder and added to the active substance. The freezing step may include freezing the active substance with liquid nitrogen. The process may further include depositing a predetermined amount of the first coating by weighing the structure and the first coating. The step of forming the second coating may include spraying the solvent to at least partially cover the first coating. The step of forming the second coating may include electrostatically depositing the solvent to at least partially cover the first coating. The step of forming the second coating may include dipping the structure with the first coating in the solvent. The step of evaporating the second coating may include heating the structure forming the first coating and the second coating. The step of evaporating the second coating may include vacuum drying the structure after the first and the second coatings have been deposited thereon. The mixture of the active substance and the polymer may be a fine powder mixture. The process may further include air brushing the structure with the first and second coating thereon to remove any debris. The step of depositing a solvent may further include placing the structure in a chamber, the chamber having a high vapor pressure of at least one solvent at a temperature one. The pre-coated device may be at a temperature two, typically lower than the temperature one, is placed in the solvent vapor and the solvent vapor condenses on the pre-coated device where it is absorbed by the pre-coating.

A bio-compatible medical device for insertion into a body prepared according to the process. A process for coating a medical device. The process may include forming a first layer on a structure, the first layer including an active substance; forming a second layer on the structure, the second layer including a bio-compatible polymer; exposing at least one of the first or the second layers to a solvent to re-flow the active substance; and drying the structure. The step of forming a first layer on the structure may include grinding the active substance into a fine powder; electrostatically charging the fine powder; discharging the structure to substantially free the structure from electrostatic charge; and depositing the fine powder on the structure. The active substance may include a polymer. The active substance may be frozen to a solid state to enhance grinding into a powder. The polymer may be frozen to a solid state to enhance grinding into a powder.

A medical device for insertion into a body prepared according to the process. The process may include forming a first layer by electrostatically binding a powder to a bio-compatible structure; forming a second layer to at least partially cover the first layer, the second layer containing at least one solvent; and evaporating the at least one solvent to cause a reflow of the first layer. The powder comprises at least one active substance in combination with at least one polymer. The structure may be a stent. The step of forming a second layer on the structure may include spray coating the structure with at least one solvent. The step of forming a second layer may include placing the structure having the first coating thereon in a chamber with a high vapor pressure of the at least one solvent having a solvent vapor temperature higher than the medical device temperature to condense on the device.

While the present invention has been described in connection with the foregoing representative embodiment, it should be readily apparent to those of ordinary skill in the art that the representative embodiment is exemplary in nature and is not to be construed as limiting the scope of protection for the invention as set forth in the appended claims.

1. A method for coating at least a portion of at least one medical device, comprising:
placing a polymer on the portion of the medical device;
placing a bioactive agent on the portion of the medical device; and
spraying, subsequent to the arranging of the polymer and the arranging of the bioactive agent, a solvent on the portion of the medical device.

2. The method of claim 1, wherein the polymer comprises one of polystyrene and polyisobutylene.

3. The method of claim 1, wherein the bioactive agent comprises paclitaxel.

4. The method of claim 1, wherein the solvent comprises at least one member chosen from a group consisting of: tetrahydrofurane, toluene, dimethylacetamide, acetone, chloroform, and alcohol.

5. The method of claim 1, wherein:
the arranging of the polymer comprises spraying the polymer in a liquid form on the medical device; and
the arranging of the bioactive agent comprises dusting the medical device with a dry powder including the bioactive agent, the arranging of the bioactive agent being performed subsequent to the arranging of the polymer.

6. The method of claim 5, further comprising drying the polymer on the medical device prior to the arranging of the bioactive agent by dusting, the drying achieved by at least one of waiting a predetermined time period, heating the medical device, and flowing a gas over the medical device.

7. The method of claim 5, wherein the arranging of the bioactive agent by dusting is performed while the polymer is wet.

8. The method of claim 5, wherein the spraying of the solvent on the portion of the medical device dissolves the dry powder including the bioactive agent.

9. The method of claim 5, wherein the spraying of the solvent on the portion of the medical device dissolves the polymer.

10. The method of claim 1, wherein the arranging of the polymer and the arranging of the bioactive agent are performed simultaneously by spraying the medical device with a solution including the bioactive agent and the polymer.

11. The method of claim 10, further comprising:
drying the solution including the bioactive agent and the polymer before spraying the solvent on the portion of the medical device;
wherein the spraying of the solvent on the portion of the medical device causes the dried solution to flow.

12. The method of claim 11, wherein the subsequent spraying of the solvent causing the dried solution to flow reduces stress in the dried solution.

13. The method of claim 11, wherein the subsequent spraying of the solvent causing the dried solution to flow smooths a surface of the dried solution.

14. The method of claim 11, wherein the subsequent spraying of the solvent causing the dried solution to flow one of repositions and redistributes the bioactive agent contained in the dried solution.

15. The method of claim 1, further comprising selecting a composition of the solvent to achieve a desired agent release profile for the medical device.

16. The method of claim 15, wherein the composition of the solvent is selected to achieve an increased agent release profile for the medical device.

17. The method of claim 16, wherein:
the solvent comprises tetrahydrofurane; and
a concentration of tetrahydrofurane is increased, thereby causing the bioactive agent to migrate to a surface of the coating.

18. The method of claim 15, wherein the composition of the solvent is selected to achieve a decreased agent release profile for the medical device.

19. The method of claim 18, wherein:
the solvent comprises toluene; and
a concentration of toluene is increased, thereby causing the polymer to migrate to a surface of the coating.

20. The method of claim 1, wherein the bioactive agent is selected from a group consisting of paclitaxel, rapamycin, everolimus and tacrolimus.

21. A medical appliance having a coating applied by a method, the method comprising:
arranging a polymer on the portion of the medical device;
arranging a bioactive agent on the portion of the medical device; and
subsequently spraying a solvent on the portion of the medical device.

22. The medical appliance of claim 21, wherein the method further comprises selecting a composition of the solvent to achieve a desired agent release profile for the medical appliance.

23. The medical appliance of claim 22, wherein the composition of the solvent is selected to achieve an increased agent release profile for the medical device.

24. The medical appliance of claim 23, wherein:
the solvent comprises tetrahydrofurane; and
a concentration of tetrahydrofurane is increased, thereby causing the bioactive agent to migrate to a surface of the coating.

25. The medical appliance of claim 22, wherein the composition of the solvent is selected to achieve a decreased agent release profile for the medical device.

26. The medical appliance of claim 25, wherein:
the solvent comprises toluene; and
a concentration of toluene is increased, thereby causing the polymer to migrate to a surface of the coating.
